(19) United States
(12) Patent Application Publication
(10) Pub. No.: US 2024/0400988 A1
(43) Pub. Date: Dec. 5, 2024

(54) Methods of Producing Astrocytes or Promoting Astrocyte Development and Cell Culture Compositions Related Thereto

(71) Applicant: Emory University, Atlanta, GA (US)

(72) Inventors: Steven Sloan, Atlanta, GA (US); Samantha Lanjewar, Atlanta, GA (US); Anna Voss, Philadelphia, PA (US)

(21) Appl. No.: 18/680,912

(22) Filed: May 31, 2024

Related U.S. Application Data
(60) Provisional application No. 63/470,105, filed on May 31, 2023.

(51) Int. Cl. C12N 5/079

(52) U.S. Cl. CPC C12N 5/0622; C12N 2501/15; C12N 2501/155; C12N 2501/20; C12N 2501/415; C12N 2501/58; C12N 2509/00

(57) ABSTRACT

This disclosure relates to methods and compositions useful for culturing astrocytes, producing astrocytes, expanding astrocytes, or promoting astrocyte development. In certain embodiments, this disclosure relates to cell culture compositions comprising components disclosed herein. In certain embodiments, this disclosure relates to methods of culturing astrocytes, promoting astrocyte development, or producing astrocytes comprising contacting a cell, stem cell, induced pluripotent cell, astrocyte, pre-astrocyte cell, or tissue with proteins BMP4, TGFβ-2, TSLP, DKK1, and NLGN1 providing astrocytes.

### CROSS-REFERENCE TO RELATED APPLICATIONS

This application claims the benefit of U.S. Provisional Application No. 63/470,105 filed May 31, 2023. The entirety of this application is hereby incorporated by reference for all purposes.

### STATEMENT REGARDING FEDERALLY SPONSORED RESEARCH OR DEVELOPMENT

This invention was made with government support under MH125956 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 23101US.xml. The XML file is 10 KB, was created on May 30, 2024, and is being submitted electronically via the USPTO patent electronic filing system.

### BACKGROUND

Astrocytes are a type of glial cell in the central nervous system (CNS) that play a role in regulating brain development, synaptic transmission, and plasticity. Recent research has highlighted the importance of astrocyte maturation in shaping neural circuitry and function. Astrocytes undergo a complex process of maturation during development, which involves changes in gene expression, morphology, and function. This process is important for the proper formation and function of synapses, neural networks, and the blood-brain barrier. Astrocyte maturation has implications for a wide range of neurological and psychiatric disorders.

In vivo neural progenitors are simultaneously exposed to a varied assortment of extrinsic cues. How these signals work to influence cell fate has been difficult to determine given the enormous potential combinations among thousands of known secreted ligands. This is further complicated by the fact that the potency of extrinsic cues may depend on many factors. The mechanisms of astrocyte expansion, migration, and maturation, remain to be fully elucidated. Thus, there is a need to identify methods for producing and promoting astrocyte development from progenitor cells.
Freeman et al. report specification and morphogenesis of astrocytes. Science, 2010, 330, 774-8 (2010).
Tchieu et al. report nuclear factor IA (NFIA) is a gliogenic switch enabling rapid derivation of functional human astrocytes from pluripotent stem cells. Nature Biotechnology, 2019, 37, 267-275.
Li et al. report generation of functional subtype astrocytes from human pluripotent stem cells. Stem Cell Reports, 2018, 11, 998-1008.
Caja et al. report transcription factor Snail regulates BMP and TGFβ pathways to control the differentiation status of glioma-initiating cells. Oncogene, 2018, 37, 2515-2531.
Tan et al. report cell adhesion molecules regulating astrocyte-neuron interactions. Curr Opin Neurobiol, 2021, 69, 170-177.
Kitic et al. report thymic stromal lymphopoietin is expressed in the intact central nervous system and upregulated in the myelin-degenerative central nervous system. Glia, 2014, 62(7): 1066-1074.
Sun et al. report neurogenin promotes neurogenesis and inhibits glial differentiation by independent mechanisms. Cell, 2001, 104, 365-76.
Luo reports that TGF-β as a key modulator of astrocyte reactivity. Biomedicines, 2022, 10(5): 1206.
Chae et al. report Dickkopfl is an immunomodulatory ligand and Wnt antagonist in pathological inflammation. Differentiation, 2019, 108: 33-39.
Yang et al. report a role of bone morphogenetic protein-2/4 in astrocyte activation in neuropathic pain. Mol Pain, 2019; 15: 1744806919892100.
Barbar et al. report CD49f is a marker of functional and reactive human iPSC-derived astrocytes. Neuron, 2020, 107(3):436-453.
See also U.S. Pat. Nos. 8,492,147, 9,018,010, and 10,557,123.

References cited herein are not an admission of prior art.

### SUMMARY

This disclosure relates to methods and compositions useful for culturing astrocytes, producing astrocytes, expanding astrocytes, or promoting astrocyte development. In certain embodiments, this disclosure relates to cell culture compositions comprising one or more components disclosed herein.

In certain embodiments, this disclosure relates to methods of culturing astrocytes, promoting astrocyte development or producing astrocytes comprising contacting a cell, stem cell, induced pluripotent cell, astrocyte, pre-astrocyte cell, or tissue with the proteins Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1) providing astrocytes, e.g., a group of cells having transcriptomic features or cell surface markers of astrocytes. In certain embodiments, proteins are in a cell culture.

In certain embodiments, this disclosure relates to methods of culturing astrocytes, promoting astrocyte development, expanding astrocytes, or producing astrocytes comprising: contacting a cell, stem cell, induced pluripotent stem cell, pre-astrocyte, or astrocyte, with a serum or serum-free growth medium comprising dorsomorphin providing induced cells; contacting the induced cells with epidermal growth factor (EGF) and fibroblast growth factor 2 (FGF2), providing secondary induced cells; contacting the secondary induced cells with brain derived neurotrophic factor (BDNF) and neurotrophin-3 (NT-3) providing neural induced cells; and contacting the neural induced cells with a combination of proteins comprised of Bone Morphogenetic Protein 4 (BMP4), Transforming Growth Factor Beta-2 (TGFβ-2), Dickkopf WNT Signaling Pathway Inhibitor 1 (DKK1), Thymic Stromal Lymphopoietin (TSLP), and Neuroligin 1 (NLGN1) providing a group of cells having transcriptomic features of astrocytes or cell surface marker associated with astrocytes.

In certain embodiments, methods disclosed herein further comprising isolating and optionally purifying an astrocyte or other cell or tissue reported herein prior to culturing/expanding an astrocyte or promoting astrocyte development or producing an astrocyte.

In certain embodiments, the group of cells having transcriptomic features or cell surface makers of astrocytes have morphological features of astrocytes.

In certain embodiments, the cell or tissue is derived from the brain, spinal cord, or neural tissue. In certain embodiments, cell is a stem cell, induced pluripotent stem cell, neural stem cell, neural-restricted intermediate progenitor, neuronal cell, radial glia (RG) cell, glial cell, or neuroepithelial cell.

In certain embodiments, the cell, astrocyte, stem cell, or induced pluripotent stem cell is derived from the brain or nerve of a subject or human patient.

In certain embodiments, this disclosure relates to cell cultures or containers comprising a combination of proteins comprised of Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1).

In certain embodiments, the cell culture comprises vitamins, amino acids, a carbohydrate fuel, a buffering agent, and optionally a pH indicator. In certain embodiments, the cell culture further comprises components in a Minimal Essential Medium (MEM) or other conventional growth medium.

In certain embodiments, the cell culture further comprises blood serum or albumin. In certain embodiments, the cell culture is serum-free comprising albumin. In certain embodiments, the cell culture further comprises insulin. In certain embodiments, the cell culture further comprises zinc and/or iron. In certain embodiments, the cell culture further comprises transferrin, selenium, ascorbic acid, an antioxidant, and/or combinations thereof or other agent(s) typically found in cell growth media.

In certain embodiments, this disclosure relates to a culture vessel or container comprising a combination of proteins comprised of Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1).

### BRIEF DESCRIPTION OF THE SEVERAL VIEWS OF THE DRAWINGS

FIGS. 1A and 1B show data indicating ligand activity depends on human cortical organoid (hCO) developmental stage.

FIG. 1A shows data following a 30-day exposure in hCOs, mouse-identified ligands 1-5 do not significantly modulate astrocyte or neuronal gene signatures.

FIG. 1B shows data indicating human-identified ligands 6-10 [Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1)] significantly increase expression of astrocyte gene signatures and decrease expression of certain neuronal genes.

FIGS. 2A-2D show data indicating synergistic and individual contributions of ligands 6-10 [Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1)].

FIG. 2A shows data on astrocyte gene signatures in the presence of each individual ligand or the full cocktail. Ligands were added every other day between day 60-90 either individually or combined together in a 5-ligand cocktail [Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1)].

FIG. 2B shows data on neuronal gene signatures in the presence of each individual ligand or the full cocktail. Ligands were added every other day between day 60-90 either individually or combined together in a 5-ligand cocktail.

FIG. 2C shows data on astrocyte gene signatures at day 120 in the presence of each individual ligand or the full cocktail. Ligands were added every other day between days 90-120 either individually or combined together in a 5-ligand cocktail.

FIG. 2D shows data on neuronal gene signatures at day 120 in the presence of each individual ligand or the full cocktail. Ligands were added every other day between days 90-120 either individually or combined together in a 5-ligand cocktail.

FIG. 3 shows data indicating an impact of candidate ligand exposures on fetal astrocytes. GW 17-20 cortices were subjected to immunopanning for CD49f+ immature astrocytes, which were cultured for 10 days in the presence or absence of the ligand cocktail [Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1)]. Astrocyte and neuronal gene signatures assessed by RNA-seq of CD490f+ cells. Fold change represents expression in ligand conditions vs control media.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of immunology, medicine, organic chemistry, biochemistry, molecular biology, pharmacology, physiology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 5% or 10%. In all cases, where the term “about” has been used to describe a value, it should be appreciated that this disclosure also supports the exact value.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, have the meaning ascribed to them in U.S. Patent law in that when applied to methods and compositions encompassed by the present disclosure refers to the idea of excluding certain prior art element(s) as an inventive feature of a claim, but which may contain additional composition components or method steps, etc., that do not materially affect the basic and novel characteristic(s) of the compositions or methods, compared to those of the corresponding compositions or methods disclosed herein.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a range of amino acids expressly specified in the claim. In certain embodiments, the disclosure contemplates that the “N-terminus of a peptide consists of an amino acid sequence,” which refers to the N-terminus of the peptide having the exact number of amino acids in the sequence and not more or having not more than a range of amino acids specified in the claim; however, the C-terminus may be connected to additional amino acids, e.g., as part of a larger peptide. Similarly, the disclosure contemplates that the “C-terminus of a peptide consists of an amino acid sequence,” which refers to the C-terminus of the peptide having the exact number of amino acids in the sequence and not more or having not more than a rage of amino acids specified in the claim; however, the N-terminus may be connected to additional amino acids, e.g., as part of a larger peptide.

The term “antioxidant” refers to molecules which inhibit reactions that are promoted by oxygen or peroxides. Antioxidants which may be used in the medium include but are not limited to reduced glutathione and ascorbic acid-2-phosphate or derivatives thereof.

The term “ingredient” refers to any compound, whether of chemical or biological origin, that can be used in cell culture media to maintain or promote the growth or proliferation of cells.

The terms “component,” “nutrient” and “ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain growth of cells ex vivo can be selected by those of skill in the art.

By “cell culture” is meant cells or tissues that are maintained, cultured, or grown in an artificial, in vitro environment.

The terms “cell culture medium,” “culture medium” and “medium formulation” refer to a nutritive solution for culturing or growing cells.

By “culture vessel” it is meant glass containers, plastic containers, or other containers of various sizes that can provide an aseptic environment for growing cells. For example, flasks, single or multiwell plates, single or multiwell dishes, or multiwell microplates can be used.

The term “container means” includes culture vessels, jars, bottles, vials, straws, ampules, and cryotubes.

The term “expand” refers to the growth and division, and not the differentiation of cells in culture.

The term “feeding” or “fluid-changing” refers to replacing the medium in which cells are cultured.

The term “combining” refers to the mixing or admixing of ingredients in a cell culture medium formulation.

The term “contacting” refers to the mixing, adding, seeding, or stirring of one or more cells with one or more compounds, solutions, media, etc.

A “serum-free” medium is a medium that contains no serum (e.g., fetal bovine serum (FBS), horse serum, goat serum, etc.).

An “astrocyte” refers to certain cells of the nervous system that have phenotypic heterogeneity. Astrocytes exist in fibrous, protoplasmic, and radial forms. Some form star-shaped cells in the brain and spinal cord. Fibrous astrocytes are usually located within white matter with long unbranched cellular segment with end-foot segments that often connect to capillary walls and express an intermediate filament glial fibrillary acidic protein (GFAP). Protoplasmic astrocytes are typically found in grey matter tissue and have highly branched tertiary segments. Radial glial astrocytes are typically found in perpendicular planes to the axes of ventricles. Subtypes of astrocytes are sometimes characterized, isolated, and/or purified by targeting cells that express transcription factors, PAX6 and NKX6.1, and/or cell surface markers CD49f, reelin, SLIT1, Integrin Beta-5, GlialCAM (or HepaCAM) adhesion protein, GLAST (Slc1a3) and ASCA-2. Contemplated techniques include cell surface fluorescent tagging, use of intracellular molecular beacons targeting mRNA, and use of magnetic beads/fluorescence-activated cell sorting methods.

The terms, “transforming growth factor beta 2,” “transforming growth factor β 2,” TGF-β-2,” “TGFbeta-2” and the like refer to a cytokine that is secreted by various cell types. There are three major TGF-β isoforms. In contrast to other members of the family, TGF β-2 does not contain a R-G-D cell attachment site motif that mediates binding to integrins Human recombinant TGF β-2 is commercially available as a disulfide-linked homodimer with each subunit containing 112 amino acids having the following sequence:

(SEQ ID NO: 1)
ALDAAYCFRNVQDNCCLRPLYIDFKRDLGWKWIHEPKGYNANFCAGACPY
LWSSDTQHSRVLSLYNTINPEASASPCCVSQDLEPLTILYYIGKTPKIEQ
LSNMIVKSCKCS.

The terms, “bone morphogenetic protein 4” and “BMP4” refer to a protein which is a natural ligand of the TGF-beta (transforming growth factor-beta). Human recombinant BMP-4 is commercially available in the form of a disulfide-linked homodimer protein (116 amino acids) having the following sequence:

(SEQ ID NO: 2)
SPKHHSQRARKKNKNCRRHSLYVDFSDVGWNDWIVAPPGYQAFYCHGDCP
FPLADHLNSTNHAIVQTLVNSVNSSIPKACCVPTELSAISMLYLDEYDKV
VLKNYQEMVVEGCGCR.

The terms, “Dickkopf WNT Signaling Pathway Inhibitor 1” and “DKK1” refer to a protein which antagonizes natural canonical Wnt signaling by inhibiting LRP5/6 interaction with Wnt. Human recombinant DKK1 is commercially available in the form as a protein (235 amino acids) having the following sequence:

(SEQ ID NO: 3)
TLNSVLNSNAIKNLPPPLGGAAGHPGSAVSAAPGILYPGGNKYQTIDNYQ
PYPCAEDEECGTDEYCASPTRGGDAGVQICLACRKRRKRCMRHAMCCPGN
YCKNGICVSSDQNHFRGEIEETITESFGNDHSTLDGYSRRTTLSSKMYHT
KGQEGSVCLRSSDCASGLCCARHFWSKICKPVLKEGQVCTKHRRKGSHGL
EIFQRCYCGEGLSCRIQKDHHQASNSSRLHTCQRH.

The terms, “Thymic Stromal Lymphopoietin” and “TSLP,” refer to a protein which interacts with a receptor composed of CRLF2 and IL7R. Human recombinant TSLP is commercially available in the form of a protein having the following sequences:

(SEQ ID NO: 4)
MYDFTNCDFEKIKAAYLSTISKDLITYMSGTKSTEFNNTVSCSNRPHCLT
EIQSLTFNPTAGCASLAKEMFAMKTKAALAIWCPGYSETQINATQAMKKR
RKRKVTTNKCLEQVSQLQGLWRRFNRPLLKQQ (132 amino acids
Met1 plus amino acids 29-159 of human TSLP
Accession No. NP_149024.1)
and
(SEQ ID NO: 5)
MFPFALLYVLSVSFRKIFILQLVGLVLTYDFTNCDFEKIKAAYLSTISKD
LITYMSGTKSTEFNNTVSCSNRPHCLTEIQSLTFNPTAGCASLAKEMFAM
KTKAALAIWCPGYSETQINATQAMKKARKSKVTTNKCLEQVS (142
amino acids of NP_149024.1) (Met1-Gln159, with
mutation Arg 127 Ala, Arg 130 Ser).

The terms, “Neuroligin 1” and “NLGN1,” refer to a protein which binds neurexins. Mature human Neuroligin 1 is a glycosylated 120 kDa molecule with a 649 amino acid (aa) extracellular domain (ECD) and a catalytically inactive cholinesterase like domain, a 21 aa transmembrane segment, and a 125 aa cytoplasmic tail. It is reported that Neuroligin 1 expression associates into dimers and tetramers. Human recombinant NLGN1 is commercially available in the form if a protein (666 amino acids) having the following sequence:

(SEQ ID NO: 6)
MALPRCTWPNYVWRAVMACLVHRGLGAPLTLCMLGCLLQAGHVLSQKLDD
VDPLVATNFGKIRGIKKELNNEILGPVIQFLGVPYAAPPTGERRFQPPEP
PSPWSDIRNATQFAPVCPQNIIDGRLPEVMLPVWFTNNLDVVSSYVQDQS
EDCLYLNIYVPTEDDIRDSGGPKPVMVYIHGGSYMEGTGNLYDGSVLASY
GNVIVITVNYRLGVLGFLSTGDQAAKGNYGLLDLIQALRWTSENIGFFGG
DPLRITVFGSGAGGSCVNLLTLSHYSEGNRWSNSTKGLFQRAIAQSGTAL
SSWAVSFQPAKYARMLATKVGCNVSDTVELVECLQKKPYKELVDQDIQPA
RYHIAFGPVIDGDVIPDDPQILMEQGEFLNYDIMLGVNQGEGLKFVENIV
DSDDGISASDFDFAVSNFVDNLYGYPEGKDVLRETIKFMYTDWADRHNPE
TRRKTLLALFTDHQWVAPAVATADLHSNFGSPTYFYAFYHHCQTDQVPAW
ADAAHGDEVPYVLGIPMIGPTELFPCNFSKNDVMLSAVVMTYWTNFAKTG
DPNQPVPQDTKFIHTKPNRFEEVAWTRYSQKDQLYLHIGLKPRVKEHYRA
NKVNLWLELVPHLHNLNDISQYTSTTTKVPSTDITFRPTRKNSVPVTSAF
PTAKQDDPKQQPSPFS.

The terms, “basic fibroblast growth factor” or “bFGF” refer to a protein that has the β-trefoil structure which binds to FGF receptor (FGFR) family members. Human recombinant bFGF is commercially available in the form of a 154 amino acid protein having the following sequence:

(SEQ ID NO: 7)
AAGSITTLPALPEDGGSGAFPPGHFKDPKRLYCKNGGFFLRIHPDGRVDG
VREKSDPHIKLQLQAEERGVVSIKGVCANRYLAMKEDGRLLASKCVTDEC
FFFERLESNNYNTYRSRKYTSWYVALKRTGQYKLGSKTGPGQKAILFLPM
SAKS.

The terms, “Epidermal growth factor” and “EGF” refer to a protein which is about a 6-kDa. Human EGF gene encodes preproprotein that is proteolytically processed to generate a peptide that functions to stimulate the division of epidermal and other cells. Human recombinant EGF is commercially available in the form of a 54 amino acid protein having the following sequence:

(SEQ ID NO: 8)
MNSDSECPLSHDGYCLHDGVCMYIEALDKYACNCVVGYIGERCQYRDLKW
WELR.

In certain embodiments, this disclosure contemplates using variants of polypeptide sequences disclosed herein having greater than 50%, 60%, 70%, 80%, 90%, 95%, or more identity. “Sequence identity” refers to a measure of relatedness between two or more nucleic acids or proteins, and it is typically given as a percentage with reference to the total comparison length. Identity calculations take into account those amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.) using default parameters. In certain embodiments, sequence “identity” refers to the number of exactly matching residues (expressed as a percentage) in a sequence alignment between two sequences of the alignment. In certain embodiments, percentage identity of an alignment may be calculated using the number of identical positions divided by the greater of the shortest sequence or the number of equivalent positions excluding overhangs wherein internal gaps are counted as an equivalent position.

Variants of proteins disclosed herein can be easily produced by a skilled artisan. One can predict functioning variants with structural similarity using computer modeling. Tests confirming inherent activity can be done using procedures outlined in the literature or in this specification. A skilled artisan would understand that one could produce a large number of operable variants that would be expected to have the desirable properties due to evolutionary conserved amino acids at specific positions. Genes are known and members share significant homologies from one species to another. The sequences are not identical as illustrated by the differences between the human and mouse sequences. Some are conserved substitutions. Some are not conserved substitutions. In order to create functioning variants, skilled artisans would not blindly try random combinations, but instead utilize computer programs to make stable substitutions. Skilled artisans would know that certain conserved substations would be desirable. In addition, a skilled artisan would not typically alter evolutionary conserved positions. See Saldano et al. “Evolutionary Conserved Positions Define Protein Conformational Diversity,” PLoS Comput Biol., 2016, 12(3):e1004775.

Guidance in determining which and how many amino acid residues may be substituted, inserted or deleted without abolishing biological activity may be found using computer programs in combination with publicly available databases well known in the art, for example, RaptorX, ESyPred3D, HHpred, Homology Modeling Professional for HyperChem, DNAStar, SPARKS-X, EVfold, Phyre, and Phyre2 software. See Kelley et al. which report the Phyre2 web portal for protein modelling, prediction and analysis, Nat Protoc., 2015, 10(6):845-858. See also Marks et al., Protein structure from sequence variation, Nat Biotechnol, 2012, 30(11):1072-1080; Mackenzie et al., Curr Opin Struct Biol, 2017, 44:161-167; Mackenzie et al., Proc Natl Acad Sci USA., 2016, 113(47), E7438-E7447 and Wei et al., Int. J. Mol. Sci., 2016, 17(12), 2118.

### Methods of Producing or Culturing Astrocytes

In certain embodiments, this disclosure relates to methods of culturing an astrocyte, promoting astrocyte development, or producing an astrocyte from a progenitor cell comprising contacting a cell, astrocyte, or tissue with Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1) providing cultured astrocytes. In certain embodiments, the cultured astrocytes have transcriptomic features of astrocytes. In certain embodiments, the cultured astrocytes have morphological features of astrocytes. In certain embodiments, the cell, astrocyte, or tissue is derived from the brain, spinal cord, or neural tissue. In certain embodiments, the cell is an induced pluripotent stem cell, neural stem cell, neural-restricted intermediate progenitor, neuronal cell, radial glia (RG) cell, glial cell, or neuroepithelial cell. In certain embodiments, the proteins are in a cell culture.

In certain embodiments, this disclosure relates to methods of culturing astrocytes, promoting astrocyte development, expanding astrocytes, or producing astrocytes comprising: contacting a cell, stem cell, astrocyte, or induced pluripotent stem cell with a serum or serum-free growth medium comprising and dorsomorphin providing induced cells; contacting the induced cells with epidermal growth factor (EGF) and fibroblast growth factor 2 (FGF2), providing secondary induced cells; contacting the secondary induced cells with brain derived neurotrophic factor (BDNF) and neurotrophin-3 (NT-3) providing neural induced cells; and contacting the neural induced cells with a combination of proteins comprised of Transforming Growth Factor Beta-2 (TGFβ-2), Bone Morphogenetic Protein 4 (BMP4), Dickkopf WNT Signaling Pathway Inhibitor 1 (DKK1), Thymic Stromal Lymphopoietin (TSLP), and Neuroligin 1 (NLGN1) providing a group of cells having transcriptomic features of astrocytes or cell surface marker associated with astrocytes.

In certain embodiments, this disclosure relates to methods of culturing astrocytes, comprising, contacting a human endothelial stem cell or induced pluripotent stem cell with a growth medium comprising, non-essential amino acids, glutamine, beta-mercaptoethanol, and dorsomorphin providing induced cells; contacting the induced cells with epidermal growth factor (EGF) and fibroblast growth factor 2 (FGF2), providing secondary induced cells; contacting the secondary induced cells with brain derived neurotrophic factor (BDNF) and neurotrophin-3 (NT-3) providing neural induced cells; and contacting the neural induced cells with a combination of proteins comprised of Transforming Growth Factor Beta-2 (TGFβ-2), Bone Morphogenetic Protein 4 (BMP4), Dickkopf WNT Signaling Pathway Inhibitor 1 (DKK1), Thymic Stromal Lymphopoietin (TSLP), and Neuroligin 1 (NLGN1) providing a group of cells having transcriptomic features of astrocytes or cell surface marker associated with astrocytes.

In certain embodiments, this disclosure relates to methods of culturing astrocytes, promoting astrocyte development, expanding astrocytes, or producing astrocytes comprising: contacting a cell, stem cell, induced pluripotent stem, pre-astrocyte, or astrocyte with a growth medium, e.g., serum-free comprising a comprising essential and non-essential amino acids, vitamins, transferrin or substitutes, insulin or insulin substitutes, trace elements, collagen precursors, and albumin preloaded with lipids, glutamine, beta-mercaptoethanol, and dorsomorphin providing induced cells; contacting the induced cells with epidermal growth factor (EGF) and fibroblast growth factor 2 (FGF2), providing secondary induced cells; contacting the secondary induced cells with brain derived neurotrophic factor (BDNF) and neurotrophin-3 (NT-3) providing neural induced cells; and contacting the neural induced cells with a combination of proteins comprised of Transforming Growth Factor Beta-2 (TGFβ-2), Bone Morphogenetic Protein 4 (BMP4), Dickkopf WNT Signaling Pathway Inhibitor 1 (DKK1), Thymic Stromal Lymphopoietin (TSLP), and Neuroligin 1 (NLGN1) providing a group of cells having transcriptomic features of astrocytes or cell surface marker associated with astrocytes.

In certain embodiments, methods disclosed herein further comprising isolating an optionally expanding and/or purifying an astrocyte prior to culturing or expanding astrocytes or promoting astrocyte development or producing an astrocyte. In certain embodiments, methods disclosed herein further comprising isolating and optionally purifying an astrocyte after culturing an astrocyte or promoting astrocyte development or producing an astrocyte as reported herein and using a cell culture comprising Bone Morphogenetic Protein 4 (BMP4), Transforming Growth Factor Beta-2 (TGFβ-2), Dickkopf WNT Signaling Pathway Inhibitor 1 (DKK1), Thymic Stromal Lymphopoietin (TSLP), and Neuroligin 1 (NLGN1) providing astrocytes or other combinations as provided herein.

In certain embodiments, this disclosure relates to methods of promoting astrocyte development, expanding an astrocyte, or producing an astrocyte comprising contacting a cell, astrocyte, or tissue with the protein Bone Morphogenetic Protein 4 (BMP4), optionally in combination with Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1), providing a group of cells having features of astrocytes.

In certain embodiments, this disclosure relates to methods of promoting astrocyte development, expanding an astrocyte, or producing an astrocyte comprising contacting a cell, astrocyte, or tissue with the proteins Bone Morphogenetic Protein 4 (BMP4), Thymic Stromal Lymphopoietin (TSLP), Dickkopf WNT Signaling Pathway Inhibitor 1 (DKK1), and Neuroligin 1 (NLGN1), optionally in combination with Transforming Growth Factor Beta-2 (TGFβ-2) providing a group of cells having features of astrocytes.

In certain embodiments, this disclosure relates to methods of promoting astrocyte development, expanding an astrocyte, or producing an astrocyte comprising contacting a cell, astrocyte, or tissue with the proteins Bone Morphogenetic Protein 4 (BMP4), Dickkopf WNT Signaling Pathway Inhibitor 1 (DKK1), Transforming Growth Factor Beta-2 (TGFβ-2) and Neuroligin 1 (NLGN1), optionally in combination with Thymic Stromal Lymphopoietin (TSLP), providing a group of cells having features of astrocytes.

In certain embodiments, this disclosure relates to methods of promoting astrocyte development, expanding an astrocyte, or producing an astrocyte comprising contacting a cell, astrocyte, or tissue with the proteins Bone Morphogenetic Protein 4 (BMP4), Thymic Stromal Lymphopoietin (TSLP), Transforming Growth Factor Beta-2 (TGFβ-2) and Neuroligin 1 (NLGN1), optionally in combination with Dickkopf WNT Signaling Pathway Inhibitor 1 (DKK1), providing a group of cells having features of astrocytes.

In certain embodiments, this disclosure relates to methods of promoting astrocyte development, expanding an astrocyte, or producing an astrocyte comprising contacting a cell, astrocyte, or tissue with the proteins Bone Morphogenetic Protein 4 (BMP4), Dickkopf WNT Signaling Pathway Inhibitor 1 (DKK1), Thymic Stromal Lymphopoietin (TSLP), Transforming Growth Factor Beta-2 (TGFβ-2) and optionally in combination with Neuroligin 1 (NLGN1), providing a group of cells having features of astrocytes.

In certain embodiments, the cell or tissue is derived from the brain, spinal cord, or neural tissue. In certain embodiments, cell is a stem cell, induced pluripotent stem cell, neural stem cell, neural-restricted intermediate progenitor, neuronal cell, radial glia (RG) cell, glial cell, or neuroepithelial cell.

In certain embodiments, the cell, astrocyte, stem cell, or induced pluripotent stem cell is derived from the brain or nerve of a subject or human patient.

In certain embodiments, the group of cells having transcriptomic features or cell surface makers of astrocytes have morphological features of astrocytes.

### Cell Cultures

In certain embodiments, this disclosure relates to cell culture compositions comprising one or more components disclosed herein. In certain embodiments, this disclosure relates to cell cultures comprising a combination of proteins comprised of Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and/or Neuroligin 1 (NLGN1). In certain embodiments the cell culture further comprises a cell, astrocyte, progenitor astrocyte, stem cell, induced pluripotent stem cell, neural stem cell, neural-restricted intermediate progenitor, neuronal cell, radial glia (RG) cell, glial cell, or neuroepithelial cell.

In certain embodiments, this disclosure relates to a culture vessel or container comprising a combination of Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1).

In certain embodiments, the cell culture comprises vitamins, amino acids, a carbohydrate fuel, a buffering agent, and optionally a pH indicator. In certain embodiments, the cell culture further comprises components in a Minimal Essential Medium (MEM) or other conventional growth medium.

In certain embodiments, the cell culture further comprises blood serum or albumin. In certain embodiments, the cell culture is a serum-free replacement. In certain embodiments, the cell culture further comprises insulin. In certain embodiments, the cell culture further comprises zinc and/or iron. In certain embodiments, the cell culture further comprises transferrin, selenium, ascorbic acid, an antioxidant, and/or combinations thereof or other agent(s) typically found in cell growth media.

The terms, “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide and/or optionally nucleotides. Typical components in a growth medium include amino acids (histidine, isoleucine, leucine, lysine, methionine, phenylalanine, threonine, tryptophan, valine, and others); vitamins such as retinol, carotene, thiamine, riboflavin, niacin, biotin, folate, and ascorbic acid; carbohydrate such as glucose, galactose, fructose, or maltose; inorganic salts such as sodium, calcium, iron, potassium, magnesium, zinc; serum; and buffering agents. Additionally, a growth media may contain phenol red as a pH indication. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganous chloride, ethanolamine, and sodium pyruvate. Minimal Essential Medium (MEM) is a term of art referring to a growth medium that contains calcium chloride, potassium chloride, magnesium sulfate, sodium chloride, sodium phosphate and sodium bicarbonate), essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin B9), choline, and myo-inositol (vitamin B8). Various growth mediums are known in the art. Dulbecco's modified Eagle's medium (DMEM) is a growth medium which contains additional components such as glycine, serine, and ferric nitrate with increased amounts of vitamins and amino acids.

Dulbecco's modified Eagle's medium (DMEM) is a growth medium which contains additional components such as glycine, serine and ferric nitrate with increased amounts of vitamins, amino acids, and glucose as provided hereafter with components (Concentration in mg/L): Amino Acids—glycine (30.0), L-arginine hydrochloride (84.0), L-cystine 2HCl (63.0), L-glutamine (584.0), L-histidine hydrochloride-H₂O (42.0), L-isoleucine (105.0), L-leucine (105.0), L-lysine hydrochloride (146.0), L-methionine (30.0), L-phenylalanine (66.0), L-serine (42.0), L-threonine (95.0), L-tryptophan (16.0), L-tyrosine disodium salt dihydrate (104.0), L-valine (94.0); Vitamins—choline chloride (4.0), D-calcium pantothenate (4.0), folic acid (4.0), niacinamide (4.0), pyridoxine hydrochloride (4.0), riboflavin (0.4), thiamine hydrochloride (4.0), i-inositol (7.2); Inorganic Salts—calcium chloride (CaCl₂) (anhyd.) (200.0), ferric nitrate (Fe(NO₃)₃:9H₂O) (0.1), magnesium sulfate (MgSO₄) (97.67), potassium chloride (KCl) (400.0), sodium bicarbonate (NaHCO₃) (3700.0), sodium chloride (NaCl) (6400.0), sodium phosphate monobasic (NaH₂PO₄—H₂O) (125.0); pH indicator—phenol Red (15.0).

Ham's F-12 medium has high levels of amino acids, vitamins, and other trace elements. Putrescine and linoleic acid are included in the formulation. as provided hereafter with components (Concentration in mg/L), NaCl (7599), KCl (223.6), Na₂HPO₄ (142), CaCl₂-2H₂O (44), MgCl₂ (122), FeSO₄·7H₂O (0.834), CuSO₄·5H₂O (0.00249), ZnSO₄·7H₂O (0.863), D-glucose (1802), Na-pyruvate (110), Phenol red (1.2), NaHCO3 (1176), L-alanine (9), L-arginine-HCl (211), L-asparagine (13.2), L-aspartic acid (13.3), L-cysteine-HCl (31.5), L-glutamine (146), L-glutamic acid (14.7), Glycine (7.5), L-histidine, —HCl·H₂O (21), L-isoleucine (4), L-leucine (13), L-lysine-HCl (36.5), L-methionine (4.47), L-phenylalanine (5), L-proline (34.5), L-serine (10.5), L-threonine (12), L-tryptophan (2), L-tyrosine (5.4), L-valine (11.7), biotin (0.0073), D-Ca-pantothenate (0.48), choline chloride (14), folic acid (1.3), i-inositol (18), nicotinic acid amide (0.037), pyridoxin-HCl (0.062), riboflavin (0.038), thiamine-HCl (0.34), vitamin B12 (1.36), hypoxanthine (4.1), thymidine (0.73), lipoic acid (0.21), linoleic acid (0.084), putrescine-2HCl (0.161).

In certain embodiments, the growth medium comprises a serum-free replacement comprising an albumin and ingredients selected from group consisting of amino acids, vitamins, a transferrin, an antioxidant, insulin, a collagen precursor, and trace elements, wherein a basal cell culture medium supplemented with said supplement is capable of supporting the growth of cells in serum-free culture. In certain embodiments, the antioxidant is selected from the group consisting of reduced glutathione and ascorbic acid an ascorbic acid-2-phosphate. In certain embodiments, the collagen precursor is selected from the group consisting of L-proline and multimers or derivatives thereof, L-hydroxyproline multimers or derivatives thereof, and ascorbic acid and multimers thereof. In certain embodiments, a transferrin substitute is an iron chelate selected from the group consisting of a ferric citrate chelate and a ferrous sulfate chelate. In certain embodiments, the transferrin substitute is ferrous sulphate in water-ethylenediaminetetraacetic acid (EDTA). In certain embodiments, the amino acid ingredient comprises amino acids selected from the group consisting of glycine, L-alanine, L-asparagine, L-cysteine, L-aspartic acid, L-glutamic acid, L-phenylalanine, L-histidine, L-isoleucine, L-lysine, L-leucine, L-glutamine, L-arginine, L-methionine, L-proline, L-hydroxyproline, L-serine, L-threonine, L-tryptophan, L-tyrosine, and L-valine, and derivatives thereof. In certain embodiments, an albumin substitute is selected from the group consisting of bovine pituitary extract, plant hydrolysate, fetal calf albumin (fetuin), egg albumin, human serum albumin (HSA), chick extract, bovine embryo extract. In certain embodiments, the trace elements are selected from the group consisting of Ag⁺, Al³⁺, Ba²⁺, Cd²⁺, Co²⁺, Cr³⁺, Ge⁴⁺, Se⁴⁺, Br, I\Mn²⁺, P, Si⁴⁺, V/Mo⁶⁺, Ni²⁺, Rb\Sn⁺ and Zr⁺. In certain embodiments, the growth medium comprises a serum-free replacement comprising glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, sodium selenite, a Ag⁺ salt, an Al³⁺ salt, a Ba²⁺ salt, a Cd²⁺ salt, a Co²⁺ salt, a Cr³⁺ salt, a Ge⁺ salt, a Se⁴⁺ salt, a Br salt, an I⁺ salt, a Mn⁺ salt, a F⁺ salt, a Si⁴⁺ salt, a V⁵⁺ salt, a Mo⁶⁺ salt, a Ni²⁺ salt, a Rb⁺ salt, a Sn²⁺ salt, and a Zr⁴⁺ salt

In certain embodiment, a growth medium is composed of a DMEM/F12 base supplemented with human serum albumin, vitamins, antioxidants, trace minerals, specific lipids, and cloned growth factors. Contemplated components are provide below as reported with associated concentration in the supplemental materials of Ludwig et al., Derivation of human embryonic stem cells in defined conditions, Nature Biotechnology volume 24, pages 185-187 (2006). Inorganic salts; calcium chloride (Anhydrous), HEPES, lithium chloride (LiCl), magnesium chloride (Anhydrous), magnesium sulfate (MgSO₄), potassium chloride (KCl), sodium bicarbonate (NaHCO₃), sodium chloride (NaCl), sodium phosphate, dibasic (Anhydrous) sodium phosphate, monohydrate (NaH₂PO₄—H₂O): Trace minerals; ferric nitrate (Fe(NO₃)₃-9H₂O), ferric sulfate (FeSO₄-7H₂O), cupric sulfate (CuSO₄-5H₂O), zinc sulfate (ZnSO₄-7H₂O), ammonium metavanadate (NH₄VO₃), manganese sulfate monohydrate (MnSO₄—H₂O), NiSO₄-6H₂O, selenium, sodium metasilicate Na₂SiO₃-9H₂O, SnCl₂, molybdic acid, ammonium salt, CdCl₂, CrCl₃, AgNO₃, AlCl₃-6H₂O, Ba(C₂H₃O₂)₂, CoCl₂-6H₂O, GeO₂, KBr, KI, NaF, RbCl, ZrOCl₂-8H₂O: Energy/fuel substrates; D-glucose, sodium pyruvate: Lipids; linoleic acid, linolenic acid, lipoic acid, arachidonic acid, cholesterol, DL-alpha tocopherol-acetate, myristic acid, oleic acid, palmitic acid, palmitoleic acid, stearic acid: amino acids; L-alanine, L-arginine hydrochloride, L-asparagine-H₂O, L-aspartic acid, L-cysteine-HCl—H₂O, L-cystine 2HCl, L-glutamic acid, L-glutamine, glycine, L-gistidine-HCl—H₂O, L-isoleucine, L-leucine, L-lysine hydrochloride, L-methionine, L-phenylalanine, L-proline, L-serine, L-threonine, L-tryptophan, L-tyrosine 2Na-2H₂O, L-valine: Vitamins; ascorbic acid, biotin, B12, Choline chloride, D-calcium pantothenate, folic acid, i-inositol, niacinamide, pyridoxine hydrochloride, riboflavin, thiamine hydrochloride: growth factors and other proteins: GABA, pipecolic acid, b-FGF, TGF-beta1, human insulin, human holo-transferrin, human serum albumin, glutathione (reduced): other components; hypoxanthine Na, phenol red, putrescine-2HCl, thymidine, 2-mercaptoethanol.

Modifications to the medium include the use of animal-sourced proteins (bovine serum albumin (BSA) and Matrigel™) and cloned zebrafish basic fibroblast growth factor (zbFGF) are reported in Ludwig et al. “Feeder-independent culture of human embryonic stem cells,” Nature Methods, volume 3, pages 637-646 (2006). Matrigel™ matrix is a solubilized basement membrane preparation extracted from the Engelbreth-Holm-Swarm (EHS) mouse sarcoma, a tumor rich in extracellular matrix proteins. Matrigel™ a partially defined extracellular matrix (ECM) extract including laminin (a major component), collagen IV, heparan sulfate proteoglycans, entactin/nidogen, and a number of growth factors such as TGF-beta1, epidermal growth factor, insulin-like growth factor, fibroblast growth factor, tissue plasminogen activator. Braam et al. report that in Matrigel™ or natural and recombinant vitronectin was effective in supporting sustained self-renewal and pluripotency in three independent human embryonic stem cells lines. Stem Cells, 2008, 26(9):2257-65.

Chen et al., report using a culture medium, defined conditions can be used for iPS cell derivation and culture, Nat Methods, 2011, 8(5): 424-429. Human ES and iPS cells can be expanded in a medium containing insulin, selenium, transferrin, L-ascorbic acid, bFGF, and TGF-β (or NODAL) in DMEM/F12 with pH adjusted with NaHCO₃. The addition of NODAL (100 ng/ml) or TGF-β1 (2 ng/ml) increased NANOG expression levels and led to consistent long-term culture stability of both human ES and iPS cells. The inclusion of either a ROCK inhibitor (HA100 or Y27632) or blebbistatin improved initial survival and supported cloning, which was further improved by the addition of transferrin and by culture in hypoxic conditions. Multiple matrix proteins, such as laminin, vitronectin and fibronectin, support human ES cell growth.

B27 supplement (Brewer et al. Journal of Neuroscience Research, 1993, 35:567-476) is reported to contain biotin, L-carnitine, corticosterone, ethanolamine, D(+)-galactose, glutathione (reduced), linoleic acid, linolenic acid, progesterone, putrescine, and retinyl acetate in addition to inorganic salts, e.g., calcium, magnesium, potassium, sodium, and iron, wherein the iron nitrate salts were at about or less than 0.1 mg/L, D-glucose, phenol red, HEPES, sodium pyruvate, proteins, and amino acids. Proteins reported include albumin, catalase, insulin, superoxide dismutase, transferrin. Amino acids reported include L-alanine (about or less than 2 mg/L), L-arginine, L-asparagine (about or less than 1 mg/L), L-cysteine (about or less than 1 or 2 mg/L), L-glutamine (absent or including), L-glutamate (absent or at low amounts, e.g., less than 5 micro grams/L), glycine, L-histidine, L-isoleucine, L-leucine, L-lysine, L-methionine, L-phenylalanine, L-proline, L-serine, L-threonine, L-tryptophan, L-tyrosine, and L-valine. Factors reported to contribute to better performance for culturing neuron cells compared to DMEM included reductions in osmolarity, glutamine, and cysteine and elimination of toxic ferrous sulphate. The total osmolarity of B27 is about 235 (e.g., 200-250) mOsm which is less than the 335 mOsm of DMEM.

As used herein a “stem cell” refers to a cell, under certain physiologic or experimental conditions, that can be induced to become tissue- or organ-specific cells with special functions. Stem cell types include embryonic stem cells, adult stem cells, and induced pluripotent stem cells. An adult stem cell or somatic cell is found among differentiated cells in a tissue or organ and can renew itself. Adult stem cells can differentiate to yield some or all of the major specialized cell types of the tissue or organ. Examples of adult stem cells include MSCs.

Embryonic stem cells (ESCs) originate from the inner cell mass of mammalian blastocysts which occur 5-7 days after fertilization. ESCs remain undifferentiated indefinitely under defined conditions and differentiate into so-called embryonic bodies when cultivated in vitro. Having pluripotency, they can differentiate into all cell types. Adult stem cells (somatic cells), such as hematopoietic, neural, and mesenchymal stem cells have an ability to become more than one cell type but do not have the ability to become any cell type.

Induced pluripotent stem cells are cells that have been naturally differentiated but exposed to chemicals and/or biologic materials in vitro (treated with reprogramming factors) that allow the cell to differentiate into a larger capacity of specialized cells, e.g., induced pluripotent stem cells (iPSCs) are differentiated cells reprogrammed to return to a pluripotent stage. Reprogrammed fully differentiated cells may be accomplished using genes involved in the maintenance of ESC pluripotency, e.g., Oct3/4, Sox2, c-Myc, Klf4, and combinations thereof. The term “induced pluripotent stem cells” refers to cells that are reprogrammed from somatic or adult stems cells to an embryonic stem cell (ESC)-like pluripotent state. See Takahashi et al. “Induction of pluripotent stem cells from mouse embryonic and adult fibroblast cultures by defined factors,” Cell, 2006, 126(4):663-676. Park et al. report reprogramming of human somatic cells to pluripotency with defined factors, Nature, 2008, 451(7175):141-146. Thus, making iPSCs in cells can typically be accomplished by in trans expression of OCT4, SOX2, KLF4 and c-MYC. Colonies appear and resemble ESCs morphologically. Alternatively, certain multipotent stem cells may require less than all of the four transcripts, e.g., cord blood CD133+ cells require only OCT4 and SOX2 to generate iPSCs. For additional guidance in generating iPSCs, see Gonzalez et al. “Methods of making induced pluripotent stem cells: reprogramming a la carte,” Nature Reviews Genetics, 2011, 12:231-242.

Induced pluripotent stem cells typically express alkaline phosphatase, Oct 4, Sox2, Nanog, and/or other pluripotency-promoting factors. It is not intended that induced pluripotent stem cells be entirely identical to embryonic cells. Induced pluripotent stem cells may not necessarily be capable of differentiating into any type of cell. TRA-1-60, TRA-1-8, or combination thereof may be used to identify human iPSCs.

In certain embodiments, this disclosure contemplates that induced pluripotent stem cells are derived from adult stem cells or mesenchymal stem cells. These terms include the cultured (self-renewed) progeny of cell populations. The term “mesenchymal stromal cells” or “mesenchymal stem cells” refers to the subpopulation of fibroblast or fibroblast-like nonhematopoietic cells with properties of plastic adherence and capable of in vitro differentiation into cells of mesodermal origin which may be derived from bone marrow, adipose tissue, Wharton's jelly in umbilical cord, umbilical cord perivascular cells, umbilical cord blood, amniotic fluid, placenta, skin, dental pulp, breast milk, and synovial membrane, e.g., fibroblasts or fibroblast-like cells with a clonogenic capacity that can differentiate into several cells of mesodermal origin, such as adipocytes, osteoblasts, chondrocytes, skeletal myocytes, or visceral stromal cells.

In certain embodiments, this disclosure contemplates that induced pluripotent stem cells are derived bone marrow derived mesenchymal stromal cells. Bone marrow derived mesenchymal stromal cells are typically expanded ex vivo from bone marrow aspirates to confluence. Certain mesenchymal stromal/stem cells (MSCs) share a similar set of core markers and properties. Certain mesenchymal stromal/stem cells (MSCs) may be defined as positive for CD105, CD73, and CD90 and negative for CD45, CD34, CD14 or CD11b, CD79a or CD19, and HLA-DR surface markers, and have the ability to adhere to plastic. See Dominici et al. “Minimal criteria for defining multipotent mesenchymal stromal cells,” The International Society for Cellular Therapy position statement, Cytotherapy, 2006, 8(4):315-317.

The term “mesenchymal stromal cells” refers to the subpopulation of fibroblast or fibroblast-like nonhematopoietic cells with properties of plastic adherence and capable of in vitro differentiation into cells of mesodermal origin which may be derived from bone marrow, adipose tissue, umbilical cord (Wharton's jelly), umbilical cord perivascular cells, umbilical cord blood, amniotic fluid, placenta, skin, dental pulp, breast milk, and synovial membrane, e.g., fibroblasts or fibroblast-like cells with a clonogenic capacity that can differentiate into several cells of mesodermal origin, such as adipocytes, osteoblasts, chondrocytes, skeletal myocytes, or visceral stromal cells. The term, “mesenchymal stem cells” refers to the cultured (self-renewed) progeny of primary mesenchymal stromal cell populations.

Bone marrow derived mesenchymal stromal cells are typically expanded ex vivo from bone marrow aspirates to confluence. Certain mesenchymal stromal/stem cells share a similar set of core markers and properties. Certain mesenchymal stromal/stem cells may be defined as positive for CD105, CD73, and CD90 and negative or low for CD45, CD34, CD14, and have the ability to adhere to plastic. See Dominici et al. Minimal criteria for defining multipotent mesenchymal stromal cells. The International Society for Cellular Therapy position statement. Cytotherapy, 2006, 8(4):315-7.

In certain embodiments, this disclosure contemplates that induced pluripotent stem cells are derived from human adipose stem cells. Sun et al. Proc Natl Acad Sci USA., 2009, 106(37):15720-15725 report induced pluripotent stem (iPS) cells can be generated from adult human adipose stem cells (hASCs) freshly isolated from patients.

Adipose tissue-derived multipotent stem cells (ADMSCs) are multipotent, undifferentiated, self-renewing progenitor cell population isolated from adipose tissue. One method to isolate ADSCs from fat tissue relies on a collagenase digestion, followed by centrifugal density gradient separation. In vitro, ADMSCs typically display a spindle-shaped morphology and lack the intracellular lipid droplets as seen in adipocytes. Isolated ADMSCs are typically expanded in monolayer cultures with a growth medium containing fetal bovine serum and/or human platelet lysate. ADMSCs have the stem cell-specific surface markers, such as CD90, CD105, CD73, and lack the expression of the hematopoietic markers CD45 and CD34.

Computational Identification of Ligand-Receptor Pairs that Drive Human Astrocyte Development

Extrinsic signaling between diverse cell types plays a role in nervous system development. Ligand binding interactions and how collections of these signals act cooperatively to affect changes in recipient cells is not entirely understood. In the developing human brain, cortical progenitor cells transition from neurogenesis to gliogenesis in a stereotyped progression that is influenced by extrinsic ligands. Therefore, experiments were performed to identify ligand combinations that act synergistically to drive gliogenesis. Using computational tools, ligand-receptor pairs were identified that are expressed at appropriate developmental stages, in relevant cell types, and whose activation is predicted to cooperatively stimulate complimentary astrocyte gene signatures. A group of five neuronally-secreted ligands were validated further evaluated for synergistic contributions to astrocyte development within both human cortical organoids and primary fetal tissue. Cooperative capabilities of these ligands were confirmed to be far greater than their individual additive capacities. It was identified that their combinatorial effects converge on AKT/mTOR signaling to drive transcriptomic and morphological features of astrocyte development.

The dynamic interactions and crosstalk between neuronal and glial cells are part of brain development. Neurons and astrocytes share a common neuroepithelial origin. The first divisions of neural stem cells called radial glia (RG) are exclusively neurogenic, either giving rise to neural-restricted intermediate progenitors or directly to young neurons. Once the bulk of neurogenesis is complete, radial glia transition to a primarily gliogenic fate referred to as the gliogenic switch. Both extrinsic and intrinsic signals can influence the gliogenic switch and the fate commitment of RG during development.

Mouse embryonic radial glia produce neurons when cultured on embryonic cortical slices but shift towards a glial fate when cultured on postnatal cortical slices. The IL-6 subfamily of molecules (CNTF, LIF, CT-1, NP and CLC) are signal-transducing coreceptors LIFRβ and gp130, and mice lacking either LIFRβ or gp130 have deficits in astrogenesis. The extrinsic factors that influence astrocyte development are thought to be secreted from newly-born cortical neurons. This creates an inherent timing mechanism whereby the extrinsic cues that are required for astrocyte formation may be supplied by the neurons whose development immediately precedes gliogenesis.

To identify cohorts of candidate ligand-receptor pairs that could influence astrocyte development, input data from a series of existing mouse and human transcriptomic datasets were applied into an in-silico framework called NicheNet. See Browaeys et al. NicheNet: modeling intercellular communication by linking ligands to target genes. Nat Methods, 2020, 17(2):159-162 A suite of transcriptional, morphological, and protein phosphorylation assays were used to demonstrate that combinatorial exposure of 5 specific ligands (TGFβ2, BMP4, DKK1, TSLP, and NLGN1) promotes astrocyte development in both an in vitro human cortical organoid model as well as primary human fetal astrocytes. In all assays, synergistic application of this ligand cocktail exhibits effects on astrocyte development that eclipse individual effects of each ligand. Additionally, specific temporal windows of RG receptivity to gliogenic ligands were identified. Protein-level readouts were used to assay candidate signaling pathways that drive astrocytic responses in the presence of the gliogenic cocktail.

Cell fate decisions during organogenesis are driven by both intrinsic and extrinsic mechanisms. While many new genomic technologies are improving our ability to disentangle intrinsic drivers of cell lineage commitment, the capacity to identify novel extrinsic signals has not grown as rapidly. This largely results from the fact that there are thousands of putative secreted molecules throughout development, each of which exhibit their own temporal dynamics. Furthermore, disentangling how these ligands act cooperatively to affect changes in recipient cells has remained an ongoing challenge. Computational tools were used to predict a group of synergistically-acting ligands on astrocyte development. Specifically, experiments indicated that TGFβ2, NLGN1, TSLP, DKK1, and BMP4 can work cooperatively to influence astrocyte development. Interestingly, with the exception of BMP4, each of these ligands exhibit minimal effects on their own. Their combinatorial influence is far greater than the sum of each individual ligand.

NicheNet for Identifying Ligand and Receptor Pairs that Influence Astrocyte Development

In humans, neurogenesis temporally precedes astrogenesis. This switch in cell fate depends on both intrinsic and extrinsic signals that act in or on RG progenitors. For an extrinsic signal to exert an astrogenic effect, it ideally meets three important criteria: first, it should be expressed by cells that are present prior to gliogenesis. Second, it should bind to cognate receptors that are expressed in astrocyte progenitors (e.g. RG) or early astrocytes. Finally, this ligand-receptor event should exert downstream changes that lead to the expression of astrocyte signature genes.

To identify ligands that meet these criteria, a computational discovery approach was applied called NicheNet. This algorithm uses transcriptomic data as input (either bulk or single cell) to identify expressed ligands and their receptors in a tissue of interest. Furthermore, NicheNet uses existing knowledge of signaling networks to predict the effects of each ligand-receptor binding event on the downstream gene expression of a set of target genes.

To identify a list of candidate ligands capable of modulating human astrocyte development, NicheNet was applied to both mouse and human datasets from the developing cortex (P3-7 for mouse and gestational week 16-19 for human). The rationale for beginning with mouse data is that it provided a source of deeply sequenced purified cell type-specific inputs, whereas human datasets are largely restricted to low-depth single cell information. Therefore, it was hypothesized that using both types of data as separate inputs would serve as a valuable screen for effectors of astrocyte development. For this study, on neuronal populations were focused as “sender cells” (ligand sources), although it is that other CNS populations can also contribute to astrocyte development. radial glia (both ventricular and outer) and immature astrocytes were defined as “receiver cells”.

NicheNet's third and final input is a target gene-set that can be used to benchmark the regulatory potential of each ligand-receptor pair. To define this target gene-set, a list of 500 astrocyte-specific genes spanning both immature and mature developmental stages were identified. From these inputs derived from the mouse dataset, a list of ligand-receptor pairs was generated that (1) are expressed in relevant cell types, (2) exhibit binding interaction, and (3) are predicted to act upstream of astrocyte-specific target genes. From this list, a group was narrowed to 5 ligands (1-5; APP, APOE, IGF1, CALB, GAS6) by focusing on candidates exhibiting complementary receptor binding and activation signatures of astrocyte genes.

For human analysis, single cell data from three separate studies of developing human fetal brain tissue was explored. Cells were assigned to specific cell types of identity based on classic cell type markers. For sender cell population, immature and mature neuronal subtypes (excitatory, inhibitory, and intermediate progenitors) were included. For receiver cells, ventricular radial glia, outer radial glia, and immature astrocytes were included. The target gene-set of interest included 500 gene human astrocyte-specific signature. This analysis yielded a separate set of candidate ligands (6-10; DKK1, BMP4, NLGN1, TGFβ2, TSLP), again selected for their complementary receptor binding and activation signatures of astrocyte genes.

As a preliminary screen of these candidate ligands, human cortical organoids (hCOs) across three hiPSC lines were exposed to a cocktail of the ligands. The hCO ligand exposures occurred over a 30-day period spanning days 60-90 in vitro, prior to the onset of gliogenesis. Ligands were added to the media every other day to maintain stable levels. To effectively readout whether this ligand cocktail influenced the balance between neuronal and glial commitment, a custom targeted RNA-seq panel were designed containing 40 astrocyte genes, 40 neuronal genes, 10 reactive astrocyte genes, and 10 control housekeeping genes. After a 30-day exposure to the ligand cocktail, targeted sequencing revealed a significant upregulation of astrocyte genes and a concomitant downregulation of neuronal genes. No control or reactive genes were significantly changed upon ligand exposure.

### Human Identified Gliogenic Ligands Influence Astrocyte Development

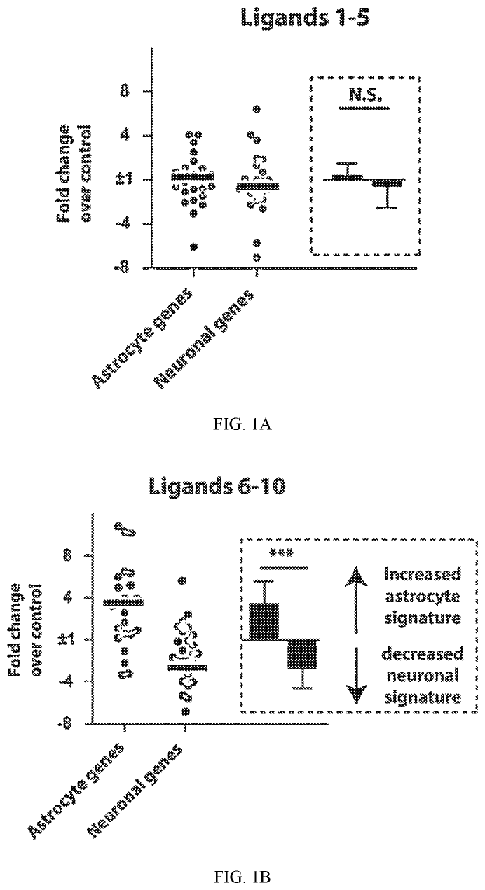

Experiments were performed to determine whether the ligands identified through the mouse data (1-5) or human single cell datasets (6-10) were specifically driving the expression changes. Therefore, the ligand cocktails were split into these two groups. Identical 30-day exposures of hCOs were performed. Again, using an astrocyte and neuronal signature gene-set as the readout, it was observed that only the human-identified ligands (6-10; DKK1, BMP4, NLGN1, TGFβ2, TSLP) induced a robust response (FIGS. 1A and 1B), whereas there was no change in the presence of the mouse-identified ligands. The effect of the group 2 (human-identified) ligands alone was also comparable to the significant effects seen with all 10 ligands added together, suggesting that the group 1 (mouse-identified) ligands were unlikely to be modulating the effects. Therefore, ligands 6-10, human-derived ligand cocktail, were used in subsequent experiments: TGFb2 (R&D Systems, Cat. 302-B2-002, 20 ng/uL), NLGN1 (Sino Biological, Cat. 11617-H08H-100, R&D Systems, Cat. AF4340, 300 ng/uL), TSLP (Sino Biological, Cat. 16135-H08H, 100 ng/uL), DKK1 (Sigma-Aldrich, Cat. SRP3258-10UG, R&D Systems, Cat. 5439-DK-010/CF, 100 ng/uL), and BMP4 (Sigma-Aldrich, Cat. H4916-10UG, R&D Systems, Cat. 314-BP-010/CF, 20 ng/uL).

The Gliogenic Switch Occurs Reproducibly Around Day 90 in hCOs

To determine if there is a temporal window during which candidate ligands most potently influence astrocyte development, experiments were performed to identify the onset of gliogenesis within hCOs. There are numerous metrics that have been used to define the initiation of astrocyte formation, each with their own caveats and advantages. Therefore, three separate assays were chosen to be as comprehensive as possible in our definition of gliogenesis. These include (1) immunohistochemistry to quantify the abundance of GFAP+ cells, (2) qPCR to quantify total GFAP mRNA within hCOs, and (3) immunopanning to pulldown HepaCAM+ astrocytes. For each metric, thresholds were set based upon values observed in human fetal tissue at gestational week 17 when gliogenesis is initiated. Next, separate differentiations of hCOs were generated across 4 hiPSC lines and assayed for each of the above outcomes at days 70, 80, 90, 100, and 110. From these assays. A temporal map of the onset of gliogenesis was created based on outcomes from each separate criterion. Remarkably, the onset of gliogenesis was reproducible and consistent across lines, differentiations, and outcome metrics at a time window between day 90-100 of hCO culture (IHC for GFAP+ cells: 92 plus or minus 9 days, qPCR for GFAP: 99 plus or minus 6 days, immunopanning for HepaCAM+ cells: 96 plus or minus 8 days).

### Ligand Exposures Affect Astrocyte Development Before and After the Gliogenic Switch

Based on this timeline of astrogenesis, experiments were performed to determine whether the ligand cocktail would exhibit differential effects when exposed to hCOs at timepoints far preceding (day 45-75), immediately before (day 60-90), and immediately after the onset of gliogenesis (day 90-120). Selection of these timepoints allows one to both assay the developmental effects of the ligands and test the temporal receptivity of RG to these signals. During exposures that lasted from day 45-75, no significant differences were found between astrocyte and neuronal gene expression. However, at the day 60-90 and day 90-120 exposures, a significant increase in astrocyte genes and concomitant decrease in neuronal gene expression were observed.

### The Cognate Receptors of the Ligands are Developmentally Regulated

Given the susceptibility of cells to respond to the ligand cocktail only at timepoints before and after the gliogenic switch, experiments were performed to determine whether the expression patterns of the cognate receptors to these ligands might correlate with developmental stages. Bulk RNA-seq of whole hCOs were performed to analyze receptor expression at various developmental timepoints (day 35, day 50, day 75, day 110). The majority of the predicted ligand-binding receptors increase in expression as hCOs approach gliogenic timepoints. Thus, the lack of significant changes in astrocyte and neuronal gene expression following ligand exposures from day 45-75 could be the result of low expression of receptors on radial glia at these timepoints. These data further confirm that the day 60-90 and day 90-120 exposures fall within a key period for astrocyte development.

### Ligands Work Synergistically, but not Individually, to Influence Astrocyte Development

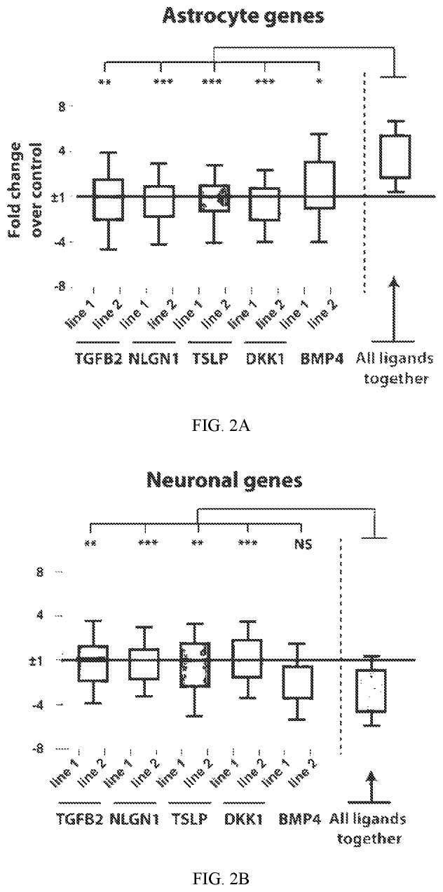

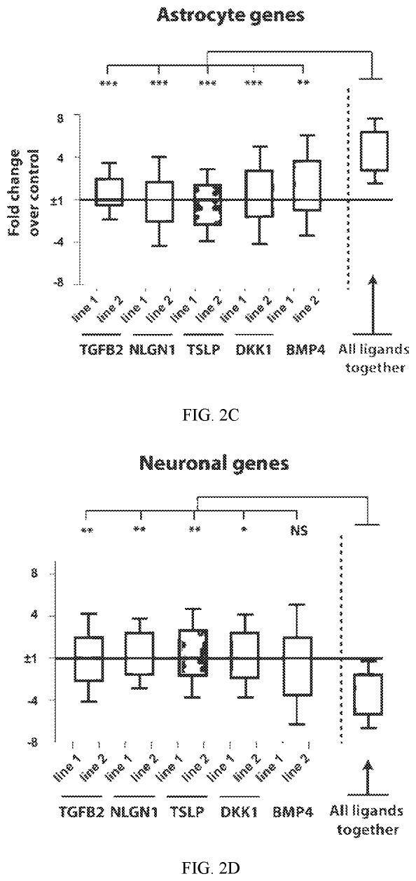

Experiments were performed to compare the impact of synergistic ligand administration versus each individual ligand on astrocyte development. Ligand exposures from day 60-90 or day 90-120, were performed adding either the ligand cocktail, or adding each ligand individually to hCOs. Of the ligands added individually, only the addition of BMP4 resulted in a significant increase in astrocyte gene signatures and concomitant decrease in neuronal gene signatures. However, at all timepoints, it was observed that the degree of astrocyte signature induction and neuronal signature depletion was most significant with all 5 ligands combined (FIGS. 2A-2D).

Experiments were designed to determine how transcriptomic changes induced by the ligand cocktail could be explained by gene changes produced by each ligand separately. Differentially expressed genes (up or down) were identified all in the presence of the ligand cocktail and at day 60-90 and 90-120, respectively (542 genes). Regardless of the administration timepoint, many cocktail-induced gene changes were also differentially expressed in at least one individual ligand condition (78%). Of these, the vast majority were perturbed in only one single ligand (57%), compared with 21% in 2 or more ligands. Interestingly, another 22% of genes (about 150) were only dysregulated when all 5 ligands were added together. Experiments were performed to determine if certain individual ligands were contributing more than others to the cocktail-induced changes. Of these transcriptomic changes, BMP4 was the predominant source and accounted for only about 35-40% of the overall gene changes observed in the ligand cocktail.

### Candidate Ligands Also Impact Astrocyte Development in Fetal Human Samples

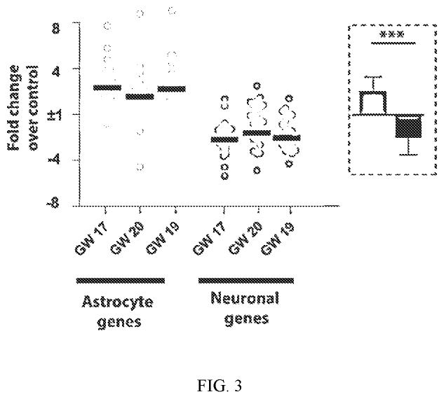

With evidence of transcriptomic changes in hCO-derived astrocyte populations, experiments were performed using primary human astrocytes. This comparison offers the added benefit of testing whether ligand perfusion into a 3D structure might impact their potency. CD49f positive astrocyte populations were purified from human fetal brain tissue and collected between 17-20 gestational weeks using immunopanning. Following purification, astrocytes were cultured in monolayer for 10-12 days with ligand exposures occurring every other day to maintain stable levels. Following ligand exposure, RNA-seq of purified cells was performed. A striking induction of astrocyte genes and downregulation of neuronal genes was again observed (FIG. 3).

### Synergistic Ligand Exposure Drives Mature Astrocyte Morphology in Purified Fetal Astrocytes

Additional experiments were performed to understand the effects of the ligand cocktail on astrocyte morphology. Investigating morphology allowed us to quantify the effects of the ligands on physical astrocyte structure, which can be a useful indicator of astrocyte maturation. Radial glia and immature astrocytes typically exhibit a more bipolar and elongated morphology, while mature astrocytes have a more branched, star-shaped morphology. Purified CD49f+ fetal cells (17-20 GW) were cultured for 10-12 days in the presence or absence of our ligand cocktail. These ligand-exposed fetal astrocyte cultures were fixed to visualize the morphology of the major branches of each GFAP positive cell. Semi-automated tracing of astrocyte processes was used to quantify the number, length, complexity, and boundary area of astrocyte branching. Using these outputs, a significantly increased number of total branches, increased number of secondary branches, decreased branch length, and decreased boundary size compared to control fetal astrocyte cultures were found.

Synergistic Ligand Exposure does not Affect Fetal Astrocyte Proliferation

Given the profound effect of the ligand exposure on inducing astrocyte gene expression, experiments were performed to whether these ligands induced proliferation of fetal astrocytes. CD49f+ fetal cells (17-20 GW) were purified and cultured with the thymidine analogue EdU for 8 days in the presence or absence of our ligand cocktail. ligand exposed fetal astrocyte cultures were fixed. Immunohistochemistry was used to visualize and quantify the percentage of proliferating cells (EdU positive/DAPI positive). No significant difference between the control and ligand exposed cells were found, suggesting the ligands do not act directly on astrocyte proliferation.

### Candidate Ligand Identification

Candidate ligands were identified using the NicheNet algorithm implemented in R version 3.6.2. Mouse data was derived from Zhang et al. Neurosci, 2014, 34(36):11929-47 and human from 3 single cell datasets of the developing fetal brain. See Eze et al. Nat Neurosci, 2021, 24, 584-594. Fan, X. et al. Cell Research, 2018, 28, 730-745. Polioudakis et al. Neuron, 2019, 103, 785-801. All input datasets were first count normalized using DESeq2. Bulk fastq files were processed by trimming using Trimmomatic™, alignment using STAR™ to mm10 and hg19, respectively, and reads were summarized using featureCounts™. Human single cell data was downloaded in count matrix format and processed using the Seurat v3 pipeline. Cell type populations were identified after using the “find markers” function and were assigned identities based on markers defined by the providing datasets. NicheNet inputs were as follows: sender cells-neuronal progenitors, immature inhibitory neurons, immature excitatory neurons, mature inhibitory neurons, mature excitatory neurons; receiver cells-radial glia, ventricular radial glia, outer radial glia, immature astrocytes. Each human single cell dataset was run through the NicheNet pipeline separately, and overlapping hits were ultimately consolidated into the final groups. Ligands with complementary predicted receptors and target genes were prioritized. A curated list of about 500 previously identified immature astrocyte genes was used as the gene set of interest to specify potential downstream targets of ligand-receptor signaling.

### Generation of Cortical Organoids

Human cortical organoids were formed from three human induced pluripotent stem cell (hiPSC) lines (8858.3, 2242.1 and 1363.1). All lines were genotyped by SNP-array to confirm genomic integrity and regularly screened for mycoplasma. iPSC colonies at 80-90% confluency were detached from culture plates using Accutase™ and were formed into 3D spheroids using AggreWell™ plates. Following 3D formation, spheroids were treated daily in neural induction media: [DMEM/F12, KSR (serum-free substitute, albumin, insulin, etc.), non-essential amino acids, Glutamax™ (L-glutamine), Penicillin-Streptomycin (antibiotics), Beta-mercaptoethanol]supplemented with dorsomorphin (Sigma, Cat. P5499-25MG, 5 μM) and 4-(4-(benzo[d][1,3]dioxol-5-yl)-5-(pyridin-2-yl)-1H-imidazol-2-yl)benzamide (SB-431542) a TGF-β receptor kinase inhibitor (TRKI). (Selleck Chemicals, Cat. S1067, 10 PM) for 6 days.

Following this treatment, organoids were treated daily with neural media supplemented with EGF and FGF2 for 10 days, and every other day for days 16-24. At day 25, organoids were treated every other day with neural media supplemented with BDNF and NT-3 to promote differentiation of progenitors. From day 43 onwards, organoids were fed every 3 days with neural media only.

### Organoid Ligand Exposures

Organoids were exposed to candidate ligands continually with media changes every other day for thirty-day periods between day 45-75, day 60-90, and day 90-120. In preliminary exposures ligands were added in two groups. Group 1 ligands were derived from mouse transcriptomic data and included: APP (20 ng/mL), APOE3(30 ng/mL), GAS6 (80 ng/mL), CALR (15 ng/mL), and IGF1 (100 ng/mL). Group 2 ligands were derived from human transcriptomic data and included: TGFβ2 (5 ng/mL), NLGN1 (50 ng/mL), TSLP (20 ng/mL), DKK1 (20 ng/mL), and BMP4 (10 ng/mL).

### Immunopanning Organoid-Derived and Human Fetal Astrocytes

Human tissue samples were obtained in compliance with policies. Astrocytes were purified from human fetal tissue between 17-20 gestational weeks. Tissue was chopped using a #10 blade and incubated in 7.5 U/ml papain at 34° C. for 45 minutes before rinsing with a protease inhibitor solution (ovomucoid). After digestion, the tissue was triturated and then the resulting single-cell suspension was added to a series of plastic petri dish pre-coated with cell type specific antibodies and incubated for 10-15 minutes each at room temperature. Unbound cells were transferred to the subsequent petri dish while the dish with bound cells was rinsed with PBS to wash away loosely bound contaminating cell types. The antibodies used include anti-CD45 to harvest and deplete microglia/macrophages, anti-Thy1 to deplete neurons, and anti-CD49f to collect astrocytes. For RNA-seq, cells were directly scraped off the panning dish with Qiazol™ (Qiagen). For cell culture and in vitro experiments, astrocytes bound to the antibody-coated dishes were incubated in a trypsin solution at 37° C. for 3-5 minutes and gently squirted off the plate. Cells were then spun and plated on poly-D-lysine-coated plastic coverslips in a Neurobasal/DMEM based serum-free medium. The media were replaced every other day for 12 days with or without ligand addition.

### Onset of Gliogenesis Experiments

Organoids from 3 previously validated hiPSC lines (8858.3, 2242.1 and 1363.1) underwent a total of 10 separate differentiations. Organoids were sampled from each differentiation at 10-day intervals from day 70 through day 110 (5 timepoints) and were administered 3 separate assays: quantitative real-time PCR (qPCR) for GFAP, immunohistochemistry IHC for GFAP, or immunopanning with anti-HepaCAM antibody. Criteria used to consider “successful” gliogenesis included a qPCR GFAP CT value less than 28, greater than 5% GFAP positive cells as a percentage of total DAPI population, and immunopanning pulldown of at least 10,000 cells (about 5% yield).

### Astrocyte Morphology Quantification

Images of GFAP positive cells were traced and analyzed using the Fiji plug-in SNT. Cells from the control and candidate ligand exposed conditions were traced using a semiautomated tracing method. Primary branches originate at the nucleus. Secondary branches extend off primary branches, and tertiary branches extend off secondary branches. The SNT software quantified the total path number, primary, secondary, and tertiary path numbers, and path length for each cell.

1. A method of culturing an astrocyte, promoting astrocyte development, or producing an astrocyte from a progenitor cell comprising contacting a cell, astrocyte, or tissue with proteins Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1) providing cultured astrocytes.

2. The method of claim 1, wherein the cultured astrocytes have transcriptomic features of astrocytes.

3. The method of claim 1, wherein the cultured astrocytes have morphological features of astrocytes.

4. The method of claim 1, wherein the cell, astrocyte, or tissue is derived from the brain, spinal cord, or neural tissue.

5. The method of claim 1, wherein the cell is an induced pluripotent stem cell, neural stem cell, neural-restricted intermediate progenitor, neuronal cell, radial glia (RG) cell, glial cell, or neuroepithelial cell.

6. The method of claim 1, wherein the proteins are in a cell culture.

7. A method of producing astrocytes comprising:
contacting a human endothelial stem cell or induced pluripotent stem cell with a growth medium comprising, non-essential amino acids, glutamine, beta-mercaptoethanol and dorsomorphin providing induced cells;
contacting the induced cells with epidermal growth factor (EGF) and fibroblast growth factor 2 (FGF2), providing secondary induced cells;
contacting the secondary induced cells with brain derived neurotrophic factor (BDNF) and neurotrophin-3 (NT-3) providing neural induced cells; and
contacting the neural induced cells with a combination of proteins comprised of Transforming Growth Factor Beta-2 (TGFβ-2), Bone Morphogenetic Protein 4 (BMP4), Dickkopf WNT Signaling Pathway Inhibitor 1 (DKK1), Thymic Stromal Lymphopoietin (TSLP), and Neuroligin 1 (NLGN1) providing a group of cells having transcriptomic features of astrocytes or cell surface marker associated with astrocytes.

8. A cell culture comprising a combination of proteins comprised of Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1).

9. The cell culture of claim 8, further comprising a cell, astrocyte, stem cell, or induced pluripotent stem.

10. The cell culture of claim 8, wherein the cell culture comprises vitamins, amino acids, carbohydrate fuel, a buffering agent, and optionally a pH indicator.

11. The cell culture of claim 8, further comprising components in a Minimal Essential Medium (MEM) or other conventional medium.

12. The cell culture of claim 8, further comprising blood serum or albumin.

13. The cell culture of claim 8, further comprising insulin, transferrin, selenium, ascorbic acid, an antioxidant, and/or combinations thereof.

14. A culture vessel or container comprising a combination of proteins Bone Morphogenetic Protein 4 (BMP4), Transforming Growth Factor Beta-2 (TGFβ-2), Thymic Stromal Lymphopoietin (TSLP), Dickkopf WNT Signaling Pathway Inhibitor 1 (DKK1), and Neuroligin 1 (NLGN1).


SEQUENCE LISTING (8 sequences)

SEQ ID NO: 1 - protein, 112 aa, synthetic construct
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 2 - protein, 116 aa, synthetic construct
SPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 3 - protein, 235 aa, synthetic construct
TLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 4 - protein, 132 aa, synthetic construct
MYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 5 - protein, 142 aa, synthetic construct
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKARKSKVTTNKCLEQVS

SEQ ID NO: 6 - protein, 666 aa, synthetic construct
MALPRCTWPNYVWRAVMACLVHRGLGAPLTLCMLGCLLQAGHVLSQKLDDVDPLVATNFGKIRGIKKELNNEILGPVIQFLGVPYAAPPTGERRFQPPEPPSPWSDIRNATQFAPVCPQNIIDGRLPEVMLPVWFTNNLDVVSSYVQDQSEDCLYLNIYVPTEDDIRDSGGPKPVMVYIHGGSYMEGTGNLYDGSVLASYGNVIVITVNYRLGVLGFLSTGDQAAKGNYGLLDLIQALRWTSENIGFFGGDPLRITVFGSGAGGSCVNLLTLSHYSEGNRWSNSTKGLFQRAIAQSGTALSSWAVSFQPAKYARMLATKVGCNVSDTVELVECLQKKPYKELVDQDIQPARYHIAFGPVIDGDVIPDDPQILMEQGEFLNYDIMLGVNQGEGLKFVENIVDSDDGISASDFDFAVSNFVDNLYGYPEGKDVLRETIKFMYTDWADRHNPETRRKTLLALFTDHQWVAPAVATADLHSNFGSPTYFYAFYHHCQTDQVPAWADAAHGDEVPYVLGIPMIGPTELFPCNFSKNDVMLSAVVMTYWTNFAKTGDPNQPVPQDTKFIHTKPNRFEEVAWTRYSQKDQLYLHIGLKPRVKEHYRANKVNLWLELVPHLHNLNDISQYTSTTTKVPSTDITFRPTRKNSVPVTSAFPTAKQDDPKQQPSPFS

SEQ ID NO: 7 - protein, 154 aa, synthetic construct
AAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 8 - protein, 54 aa, synthetic construct
MNSDSECPLSHDGYCLHDGVCMYIEALDKYACNCVVGYIGERCQYRDLKWWELR